(19) United States
(12) Patent Application Publication
(10) Pub. No.: US 2018/0088073 A1
(43) Pub. Date: Mar. 29, 2018

(54) GAS SENSOR AND METHOD OF MANUFACTURE THEREOF

(71) Applicants: Rohm and Haas Electronic Materials LLC, Marlborough, MA (US); Rohm and Haas Electronic Materials Korea Ltd., Cheonan-si (KR)

(72) Inventors: Eui Hyun Ryu, Yongin (KR); Peter Trefonas, III, Medway, MA (US); Bok-Hee Lee, Osan (KR)

(21) Appl. No.: 15/715,564

(22) Filed: Sep. 26, 2017

Related U.S. Application Data
(60) Provisional application No. 62/400,008, filed on Sep. 26, 2016.

(51) Int. Cl. G01N 27/407; H01L 41/113; G01N 33/00; G01N 29/02

(52) U.S. Cl. CPC G01N 27/407; H01L 41/1134; G01N 33/0009; G01N 33/0006; G01N 29/022; G01N 33/0027

(57) ABSTRACT

Disclosed herein is a gas sensor comprising a substrate; a first polymeric layer having a first surface and a second surface disposed on the substrate; where the first surface contacts the substrate and where the second surface is opposed to the first surface and has a higher surface area than the first surface; where the first polymeric layer comprises repeat units that have a deprotected hydrogen donor; and a second polymeric layer disposed on the first polymeric layer; where the second polymeric layer is derived from a repeat unit that comprises a hydrogen acceptor.

### CROSS REFERENCE TO RELATED APPLICATIONS

This application is a Non-Provisional application, which claims the benefit of U.S. Provisional Application No. 62/400,008, filed Sep. 26, 2016, the entire contents of which is hereby incorporated by reference.

### BACKGROUND

This disclosure relates to a gas sensor and to a method of manufacture thereof.

Gas sensors are used for detecting the presence of hazardous, unwanted and disagreeable gases in the home as well as in industry. Examples of such gases are carbon monoxide, carbon dioxide, formaldehyde, hydrogen sulfide, amines, ozone, ammonia, benzene, and so on. In order to detect these hazardous gases, functionalized polymers that have display weak interactions such as hydrogen bonding, van der Waals interactions, π-π interaction and electrostatic interactions with the gases are used. These functionalized polymers are generally coated on the surface of sensor electrodes, which are in direct contact with piezo-electric sensors used in the chemical sensor.

During this detection process, a weight change from the captured hazardous, unwanted and disagreeable gases are transformed to an electric current by a piezo-electric process. Therefore, the sensitivity of the gas sensor is dependent on the amount of gas that contacts the detection surface of the sensor. In order to improve detection capabilities, it is therefore desirable to increase the surface area of the contact surface of the sensor.

### SUMMARY

Disclosed herein is a gas sensor comprising a substrate; a first polymeric layer having a first surface and a second surface disposed on the substrate; where the first surface contacts the substrate and where the second surface is opposed to the first surface and has a higher surface area than the first surface; where the first polymeric layer comprises repeat units that have a deprotected hydrogen donor; and a second polymeric layer disposed on the first polymeric layer; where the second polymeric layer is derived from a repeat unit that comprises a hydrogen acceptor.

Disclosed herein too is a method of manufacturing a gas sensor comprising disposing upon a substrate a first polymeric layer having a first surface and a second surface; where the first surface contacts the substrate and where the second surface is opposed to the first surface and has a higher surface area than the first surface; where the first polymeric layer comprises repeat units that have a deprotected hydrogen donor; and disposing upon the first polymeric layer a second polymeric layer; where the second polymeric layer is derived from a repeat unit that comprises a hydrogen acceptor.

Disclosed herein too is a method of detecting a gas comprising contacting a gas sensor with a gaseous molecule; where the gas sensor comprises a substrate; a first polymeric layer having a first surface and a second surface disposed on the substrate; where the first surface contacts the substrate and where the second surface is opposed to the first surface and has a higher surface area than the first surface; where the first polymeric layer comprises repeat units that have a deprotected hydrogen donor; and a second polymeric layer disposed on the first polymeric layer; where the second polymeric layer is derived from a repeat unit that comprises a hydrogen acceptor; and forming at least one of a hydrogen bond, a van der Waals interaction, a π-π interaction or an electrostatic interaction between the gas molecule and the second polymeric layer; and determining an identity of the gas molecule based on a weight difference of the sensor prior to and after the forming of the hydrogen bond, the van der Waals interaction, the π-π interaction and the electrostatic interaction.

### BRIEF DESCRIPTION OF THE FIGURES

FIG. 1 depicts an exemplary exploded view of a gas sensing device assembly;

FIG. 2(A) depicts one exemplary embodiment of the substrate with the first and second polymeric layers disposed thereon;

FIG. 2(B) depicts another exemplary embodiment of the substrate with the first and second polymeric layers disposed thereon;

FIG. 3 depicts one method of manufacturing the sensing element; and

FIG. 4 depicts another method of manufacturing the sensing element.

### DETAILED DESCRIPTION

Disclosed herein is a gas sensor and a sensor element for the gas sensor that improves the sensitivity of detection of undesirable hazardous gases in the atmosphere. The sensor element comprises a substrate on which is disposed a first polymeric layer. In an embodiment, the substrate is a piezoelectric crystal that converts a sensor element weight change into an electrical signal. The first polymeric layer has a first surface that contacts the substrate and a second surface that is opposed to the first surface which has a higher surface area than the first surface. In an exemplary embodiment, the second surface has a textured surface. The texture increases the surface area significantly over the same surface when it is not textured.

In an embodiment, the first polymeric layer comprises repeat units that have a deprotected hydrogen donor. Disposed on the first polymeric layer is a second polymeric layer. The second polymeric layer has repeat units that comprise a hydrogen acceptor.

When a gas molecule contacts a free surface (where the free surface is a surface that contacts the ambient atmosphere) of the second polymeric layer, it interacts with the free surface by hydrogen bonding, van der Waals interactions, π-π interactions, electrostatic interactions, or a combination thereof, which increases the weight of the sensor element. The piezoelectric crystal converts the weight difference of the sensing element to an electrical signal, which is then used to determine the identity of the hazardous gas.

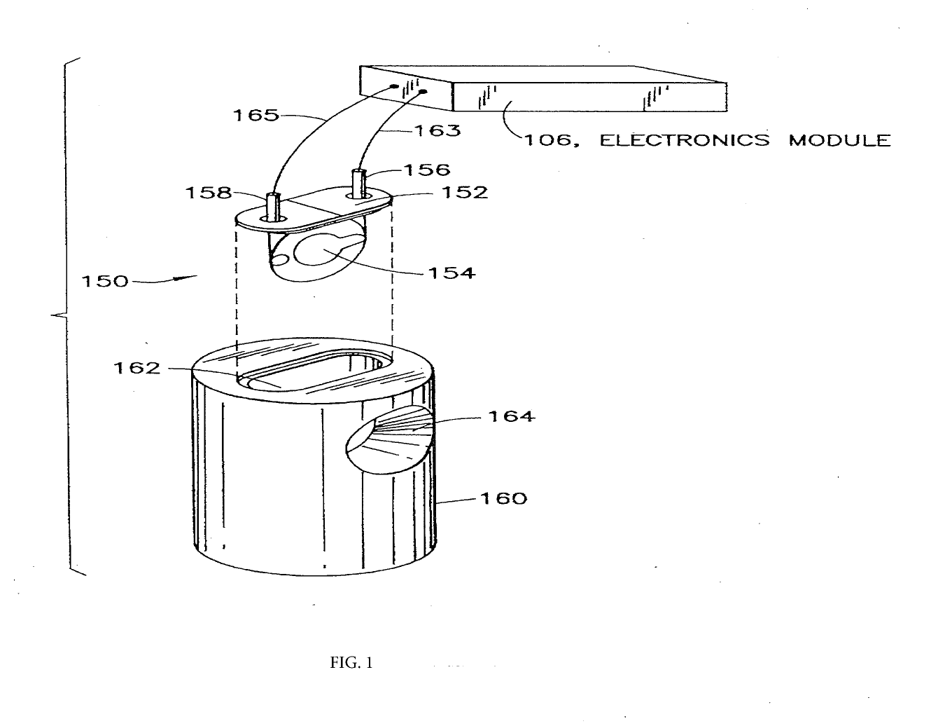

The FIG. 1 shows an exploded view of a gas sensing device assembly 100 that comprises a sensor element 150 and a housing 160. The sensor element 150 comprises a substrate 154 which is coated with the first polymeric layer and the second polymeric layer 155 (hereinafter multilayered coating 155) that interacts with a fluid component of interest to yield an interaction product of differing mass characteristic than the original multilayered coating. In an embodiment, the substrate 154 comprises a piezoelectric crystal.

The substrate 154 with the multilayered coating disposed thereon (also referred to herein as a coated substrate) is mounted on the plug member 152, with the respective leads of the substrate 154 protruding exteriorly of the plug member when the plug member is engaged with the housing 160 with the coated substrate extending into the cavity 162. The housing 160 features an opening 164 by which a gas can flow into the cavity 162 containing the sensor element 150. Although not shown in the front perspective view of the FIG. 1, the housing 160 has another opening therein, opposite opening 164 and in register with such opening, for discharge from the housing of the fluid component that flows past the sensor element 150.

The leads 156 and 158 of the sensor element 150 may contact suitable electronics as shown schematically as electronics module 166 in the FIG. 1, by which the presence a concentration of the hazardous gas species can be detected. The electronics module 166 contacts the sensor element leads 156 and 158 by wires 163 and 165, respectively.

Electronics module 166 provides the functions of (i) sampling the output resonant frequency of the piezoelectric crystal while an oscillating electric field is applied thereto, (ii) determining the change in resonant frequency from the fundamental resonant frequency incident to the formation of the interaction product when the sensor material interacts with the gas species in the fluid being monitored, and (iii) generating an output indicative of the presence of the gas species in such fluid.

In a specific embodiment of the sensor assembly shown in the FIG. 1, the housing 160 may comprise a metal or plastic housing which has the cavity 162 machined into it for the insertion of the sensor element 150, as well as two feedthrough openings (opening 164 and the opposite opening not shown in FIG. 1) for the gas being monitored to flow through the sensor. In the body of this housing is the flow restricting orifice. Front end driver electronics are plugged directly onto the legs (leads 156 and 158) of the sensor assembly.

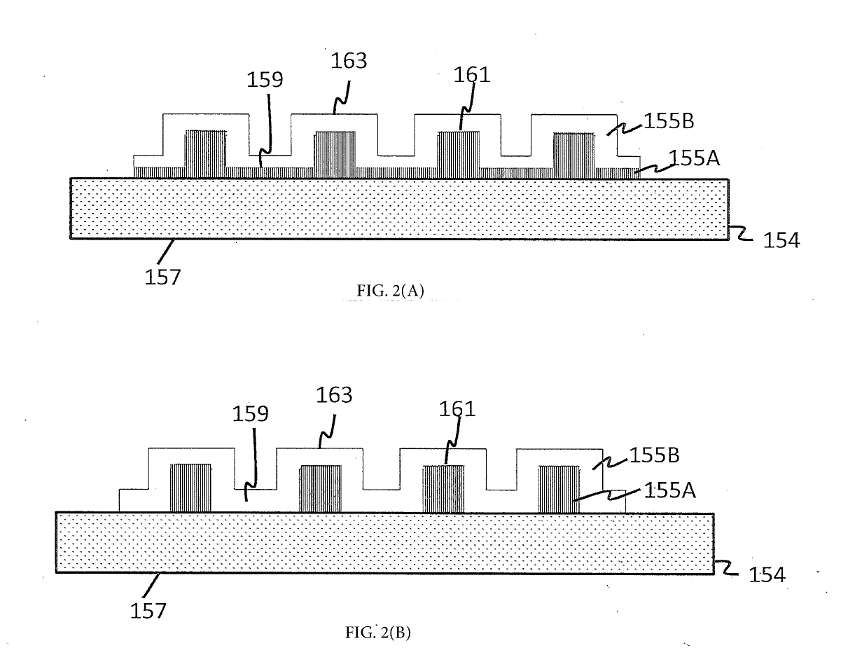

The FIGS. 2(A) and 2(B) show the substrate 154 with the multilayered coating 155 disposed thereon. Suitable substrates 154 are those that display piezoelectric properties. Examples of such substrates are quartz, berlinite (AlPO₄), topaz, tourmaline-group minerals, lead titanate (PbTiO₃), Langasite (La₃Ga₅SiO₁₄), a quartz-analogous crystal; gallium orthophosphate (GaPO₄), also a quartz-analogous crystal; lithium niobate (LiNbO₃), lithium tantalate (LiTaO₃), barium titanate (BaTiO₃), lead zirconate titanate (Pb[ZrₓTi₁₋ₓ]O₃ with 0≦x≦1)—more commonly known as PZT, potassium niobate (KNbO₃), sodium tungstate (Na₂WO₃), Ba₂NaNb₅O₅, Pb₂KNb₅O₁₅, zinc oxide (ZnO), polyvinylidene fluoride (PVDF), or the like, or a combination thereof.

The multilayered coating 155 comprises the first polymeric layer 155A and the second polymeric layer 155B. The first polymeric layer 155A has opposing surfaces 157 and 159, where surface 157 (hereinafter the first surface 157) contacts the substrate 154. The second surface 159 is textured and contacts the second polymeric layer 155B. In an embodiment, the second polymeric layer 155B has a first surface 161 and a second surface 163. The first surface 161 of the second polymeric layer 155B contact the second surface 159 of the first polymeric layer 155A. In an embodiment, the second surface 163 of the second polymeric layer 155B is parallel to the second surface 159 of the second polymeric layer 155B.

The first polymeric layer 155A comprises repeat units that have a deprotected hydrogen donor. It is desirable for the deprotected hydrogen donor to chemically interact with the polymeric units that constitute the second polymeric layer 155B in order to prevent macrophase separation of the first polymeric layer 155A from the second polymeric layer 155B. The polymer used in the first polymeric layer 155A may be a thermoplastic polymer that comprises either a homopolymer, a copolymer, or a combination thereof. The copolymer may be a block copolymer (e.g., a diblock copolymer or a triblock copolymer), an alternating copolymer, a random copolymer, a gradient copolymer, a graft copolymer, a star block copolymer, an ionomer, or a combination comprising at least one of the foregoing polymers.

The homopolymer or copolymer of the first polymeric layer 155A comprises a deprotected hydrogen donor that interacts (e.g., by forming a bond) with a hydrogen acceptor group in the second polymeric layer 155B. The deprotected hydrogen donor is obtained by deprotecting a protected hydrogen donor (e.g., a protected acid group or protected alcohol group) that is present in the homopolymer or copolymer of the first polymeric layer 155A. The protected hydrogen donor group (also referred to as a blocked hydrogen donor group) generally comprises a protected acid group or a protected alcohol group. The acid and/or alcohol group is protected by a moiety (e.g., a decomposable group) that can be deprotected by exposure to an acid, to an acid generator (such as thermal acid generator or a photoacid generator), to thermal energy or to electromagnetic radiation.

Suitable acid decomposable groups that may be used for the blocking is a C₄₋₃₀ tertiary alkyl ester. Exemplary C₄₋₃₀ tertiary alkyl groups include 2-(2-methyl)propyl (“t-butyl”), 2-(2-methyl)butyl, 1-methylcyclopentyl, 1-ethylcyclopentyl, 1-methylcyclohexyl, 1-ethylcyclohexyl, 2-methyladamantyl, 2-ethyladamantyl, or a combination comprising at least one of the foregoing. In a specific embodiment, the acid decomposable group is a t-butyl group or an ethylcyclopentyl group.

Additional decomposable groups for protecting carboxylic acids include substituted methyl esters such as methoxymethyl, tetrahydropyranyl, tetrahydrofuranyl, 2-(trimethylsilyl)ethoxymethyl, benzyloxymethyl, and the like; 2-substituted ethyl esters, such as 2,2,2-trichloroethyl, 2-haloethyl, 2-(trimentylsilyl)ethyl, and the like; 2,6-dialkylphenyl esters such as 2,6-dimethylphenyl, 2,6-diisopropylphenyl, benzyl, and the like; substituted benzyl esters such as triphenylmethyl, p-methoxybenzyl, 1-pyrenylmethyl, and the like; silyl esters such as trimethylsilyl, di-t-butylmethylsilyl, triisopropylsilyl, and the like.

Examples of groups that can be decomposed by electromagnetic radiation to form a free carboxylic acid or alcohol include:

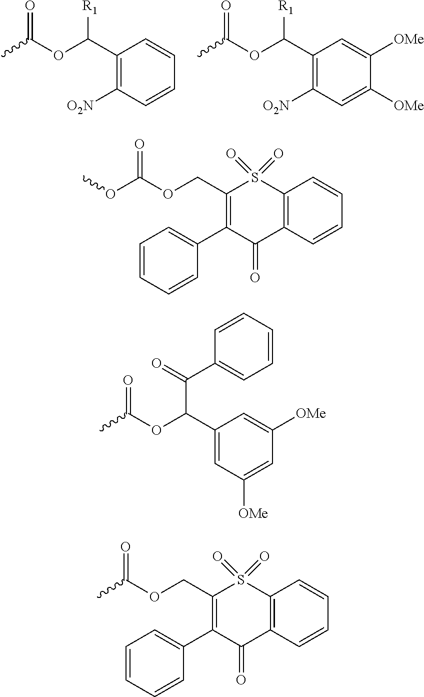

Examples of groups that can be decomposed by electromagnetic radiation to form a free amine include:

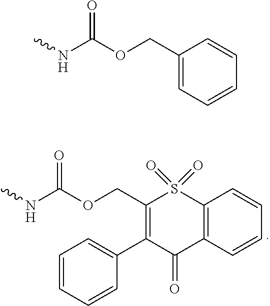

Additional protecting groups and methods to decompose them are known in the art of organic chemistry and are summarized by Greene and Wuts in “Protective groups in organic synthesis”, Third Edition, John Wiley & Sons, Inc., 1999.

When the first polymer layer 155A comprises a copolymer, the first polymer comprises the blocked hydrogen donor while the second polymer may be a neutral polymer that is incompatible with the first polymer.

The first block (also referred to as a blocked hydrogen donor) contains a protected acid group and/or a protected alcohol group. The acid and/or alcohol group is protected by a moiety that can be deprotected by exposure to an acid, to an acid generator (such as thermal acid generator or a photoacid generator), to thermal energy or to electromagnetic radiation. Suitable acid decomposable groups are listed above.

The temperature of decomposition of the protected group is 100 to 250° C. The electromagnetic radiation comprises UV radiation, infrared radiation, xrays, electron beam radiation, and the like. Exemplary protected acid groups are shown below in the formulas (1)-(9).

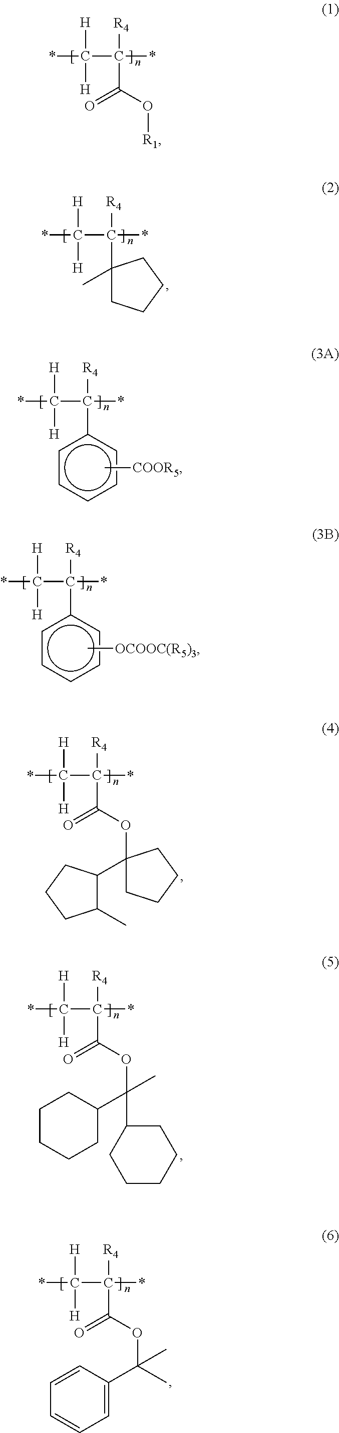

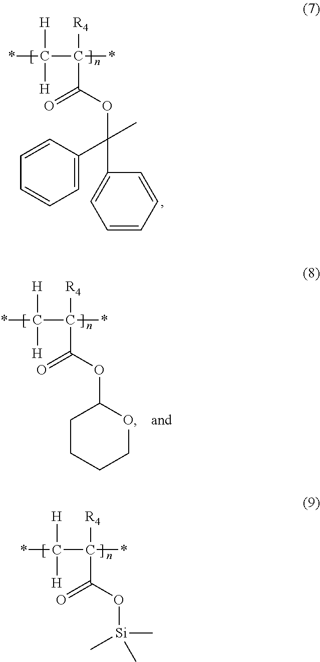

where n is the number of repeat units, R₁ is a C₁ to C₃₀ alkyl group, preferably a C₂ to C₁₀ alkyl group, R₄ is the formulas (7) through (12D) is a hydrogen, a C₁ to C₁₀ alkyl, and where R₅ is a hydrogen or a C₁ to C₁₀ alkyl. In the formula (8), the oxygen heteroatom may be located at either the ortho, meta or para position.

Other acid groups that may be protected can include phosphoric acid groups and sulfonic acid groups. Shown below are blocks that contain a sulfonic acid group and a phosphoric acid group that may be used in the block copolymer of the first polymeric layer 155A.

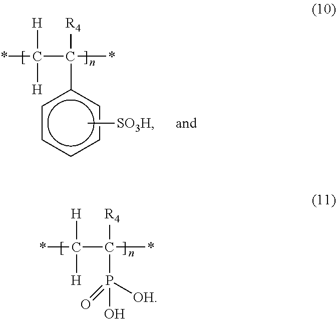

Suitable oxygen-containing groups can form a hydrogen bond with a deprotected alcohol group at the surface of the resist pattern. Useful oxygen-containing groups include, for example, ether and alcohol groups. Suitable alcohols include, for example, primary hydroxyl groups such as hydroxymethyl, hydroxyethyl, and the like; secondary hydroxyl groups such as 1-hydroxyethyl, 1-hydroxypropyl, and the like; and tertiary alcohols such as 2-hydroxypropan-2-yl, 2-hydroxy-2-methylpropyl, and the like; and phenol derivatives such as 2-hydroxybenzyl, 3-hydroxybenzyl, 4-hydroxybenzyl, 2-hydroxynaphthyl, and the like. Useful ether groups include, for example, methoxy, ethoxy, 2-methoxyethoxy, and the like. Other useful oxygen-containing groups include diketone functionalities such as pentane-2,4-dione, and ketones such as ethanone, butanone, and the like.

Examples of the protected alcohol block are shown in the Formulas (12) and (13),

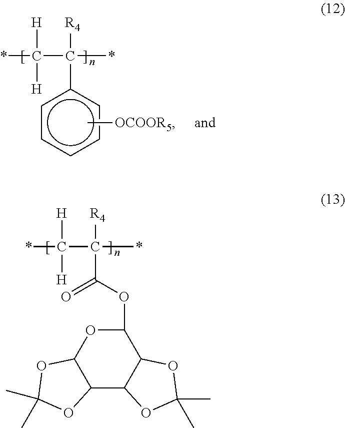

where n is the number or repeat units, R₄ is the Formula (12) is a hydrogen or a C₁ to C₁₀ alkyl, and where R₅ is a hydrogen or a C₁ to C₁₀ alkyl.

In addition to the polymers shown in the Formulas (1) through (13), other monomers that may be converted to polymers and used either as homopolymers or as part of a copolymer in the first polymeric layer 155A are shown, for example, below.

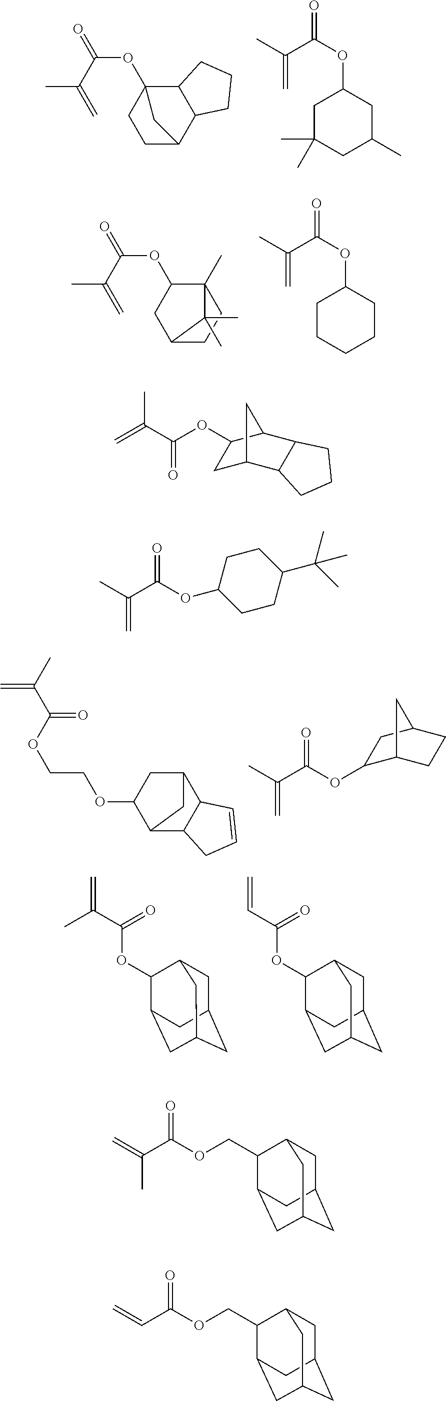

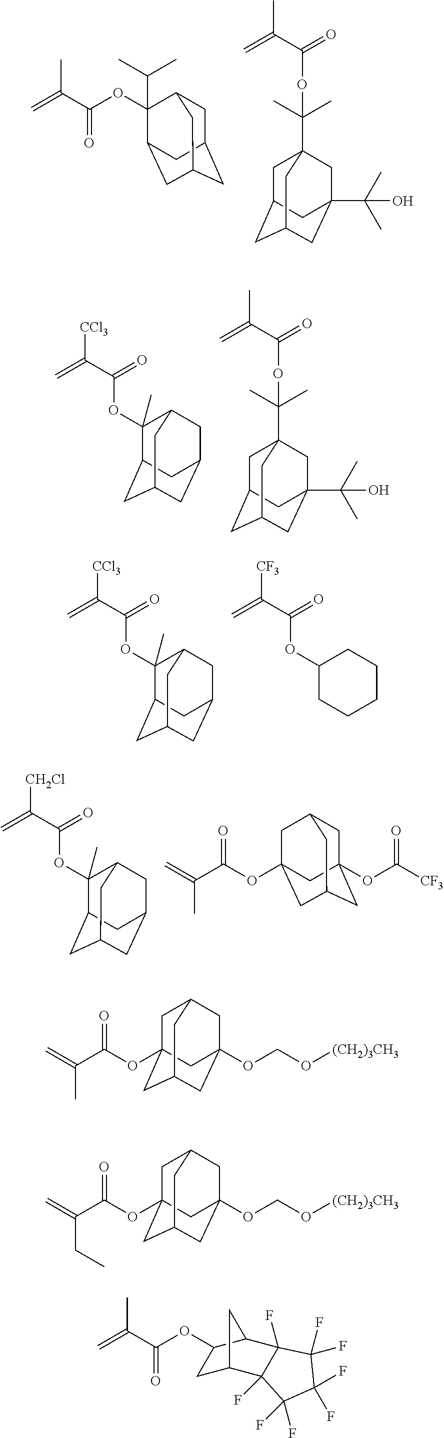

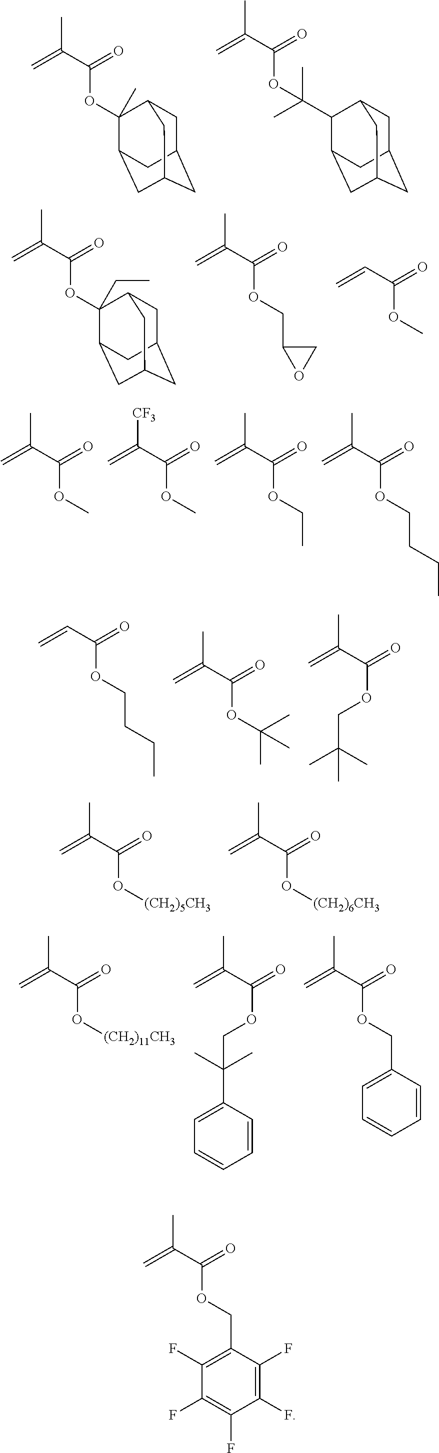

When the first polymer used in the first polymeric layer 155A is a homopolymer it has a weight average molecular weight average of 1,000 to 100,000 grams per mole, preferably 5,000 to 30,000 grams per mole.

When the first polymer used in the first polymeric layer 155A is part of a copolymer it has a weight average molecular weight average of 1,000 to 100,000 grams per mole, preferably 5,000 to 30,000 grams per mole.

Examples of the second polymer of the copolymer used in the first polymeric layer 155A comprise polystyrene, polyacrylates, polyolefins, polysiloxanes, polycarbonates, polyacrylics, polyesters, polyamides, polyamideimides, polyarylates, polyarylsulfones, polyethersulfones, polyphenylene sulfides, polyvinyl chlorides, polysulfones, polyimides, polyetherimides, polytetrafluoroethylenes, polyetherketones, polyether etherketones, polyether ketone ketones, polybenzoxazoles, polyphthalides, polyanhydrides, polyvinyl ethers, polyvinyl thioethers, polyvinyl ketones, polyvinyl halides, polyvinyl nitriles, polyvinyl esters, polysulfonates, polysulfides, polythioesters, polysulfonamides, polyureas, polyphosphazenes, polysilazanes, or the like, or a combination thereof.

An exemplary second polymer of the copolymer used in the first polymeric layer 155A is derived from a vinyl aromatic monomer. The vinyl aromatic monomer of the second block is preferably of the following general formula (14):

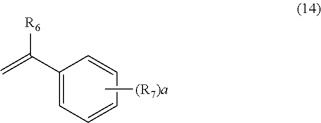

wherein: R₆ is chosen from hydrogen and C₁ to C₃ alkyl or haloalkyl such as fluoro-, chloro-, iodo- or bromoalkyl, with hydrogen being typical; R₇ is independently chosen from hydrogen, halogen (F, Cl, I or Br), and optionally substituted alkyl such as optionally substituted C₁ to C₁₀ linear or branched alkyl or C₃ to C₈ cyclic alkyl, optionally substituted aryl such as C₅ to C₂₅, C₅ to C₁₅ or C₅ to C₁₀ aryl or C₆ to C₃₀, C₆ to C₂₀ or C₆ to C₁₅ aralkyl, and optionally including one or more linking moiety chosen from —O—, —S—, —C(O)O— and —OC(O)—, wherein two or more R₂ groups optionally form one or more rings, for example, fused rings such as naphthyl, anthracenyl and the like; and a is an integer from 0 to 5.

Suitable vinyl aromatic monomers of the formula (14) include monomers chosen, for example, from the following:

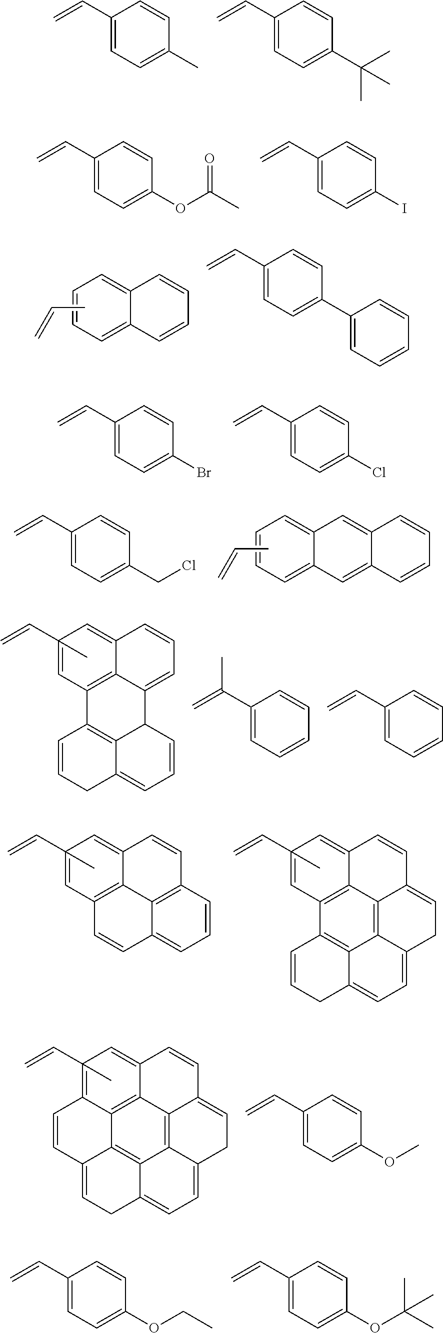

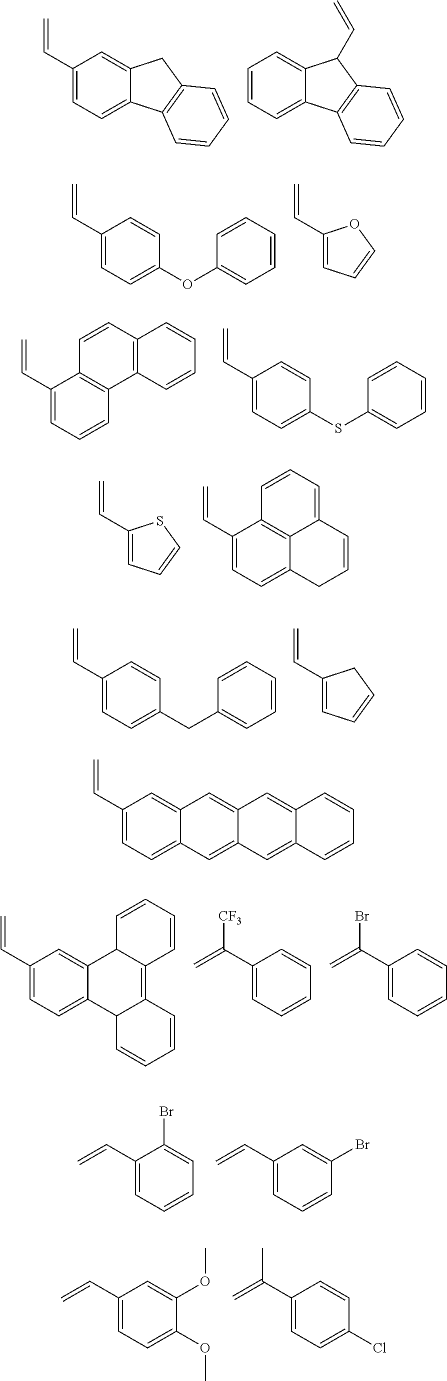

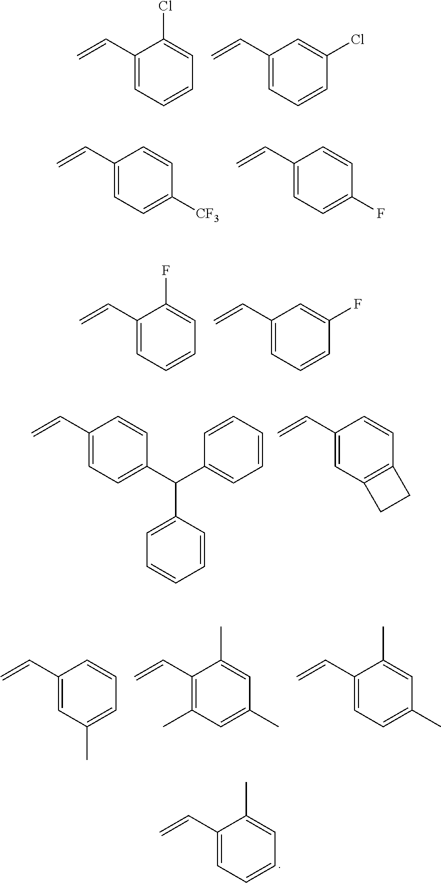

When the second polymer used in the first polymeric layer 155A is part of a copolymer it has a weight average molecular weight average of 1,000 to 100,000 grams per mole, preferably 3,000 to 30,000 grams per mole.

The second polymeric layer 155B generally comprises a polymer derived from repeat units that contain a hydrogen acceptor and a sensing acceptor. In an embodiment, the hydrogen acceptor is the same as the sensing acceptor. In another embodiment, the hydrogen acceptor is different from the sensing acceptor. The polymer used in the second polymeric layer 155B can undergo an interaction with the polymer used in the first polymeric layer 155A to prevent the first polymeric layer 155A from macrophase separating from the second polymeric layer 155B. The hydrogen acceptor is operative to undergo hydrogen bonding, van der Waals interactions, π-π interactions, electrostatic interactions, or a combination thereof, with the deprotected hydrogen donor of the first polymeric layer 155A. The sensing acceptor is also operative to undergo hydrogen bonding, van der Waals interactions, π-π interactions, electrostatic interactions, or a combination thereof, with hazardous, unwanted or disagreeable gas molecules present in the ambient atmosphere surrounding the sensor.

The hydrogen acceptor containing polymer used in the second polymeric layer 155B may be a homopolymer or a copolymer. In an embodiment, the hydrogen acceptor containing polymer can be a random copolymer or a block copolymer. When the hydrogen acceptor containing polymer used in the second polymeric layer 155B is a copolymer, both polymers generally comprise repeat units that have hydrogen acceptors. In an embodiment, when the polymer used in the second polymeric layer 155B is a homopolymer, the hydrogen acceptor may also act as the sensing acceptor. In another embodiment, when the polymer used in the second polymeric layer 155B is a copolymer, one of the repeat units may function as the hydrogen acceptor, while the other may function as the sensing acceptor. Alternatively, one of the repeat units of the copolymer may function as both the hydrogen acceptor and the sensing acceptor, while the other repeat unit performs another function.

The hydrogen acceptor containing polymer generally comprises a nitrogen-containing group. The sensing acceptor comprises nitrogen-containing groups, aliphatic or aromatic groups, or halogenated aliphatic or aromatic groups. Suitable nitrogen-containing groups can form an ionic bond with an acid group at the surface of the polymeric layers 155A and 155B. Useful nitrogen-containing groups include, for example, amine groups and amide groups, for example, primary amines such as amine, secondary amines such as alkylamines including N-methylamine, N-ethylamine, N-t-butylamine, and the like, tertiary amines such as N,N-dialkylamines including N,N-dimethylamine, N,N-methylethylamine, N,N-diethylamine, and the like. Useful amide groups include alkylamides such as N-methylamide, N-ethylamide, N-phenylamide, N,N-dimethylamide, and the like. The nitrogen-containing groups can also be part of a ring, such as pyridine, indole, imidazole, triazine, pyrrolidine, azacyclopropane, azacyclobutane, piperidine, pyrrole, purine, diazetidine, dithiazine, azocane, azonane, quinoline, carbazole, acridine, indazole, benzimidazole, and the like. Preferred nitrogen containing groups are amine groups, amide groups, pyridine groups, or a combination thereof. In an embodiment, the amine in the second polymeric layer 155B forms an ionic bond with the free acid at the surface of the first polymeric layer 155A to anchor the second polymeric layer 155B.

In an embodiment, the repeat unit of the polymer used in the second polymeric layer 155B contains a hydrogen acceptor. In an embodiment, the hydrogen acceptor comprises a nitrogen containing group. Examples of hydrogen acceptor containing polymer that comprise a nitrogen containing group are shown below in the Formulas (15) to (20),

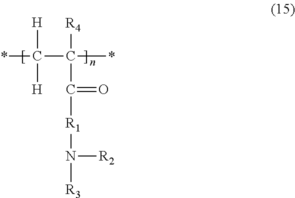

where n is the number of repeat units, and where R₁ is a C₁ to C₃₀ alkyl group, preferably a C₂ to C₁₀ alkyl group, R₂ and R₃ can be the same or different and can be hydrogen, a hydroxyl, a C₁ to C₃₀ alkyl group, preferably a C₁ to C₁₀ group, and wherein R₄ is a hydrogen or a C₁ to C₃₀ alkyl group.

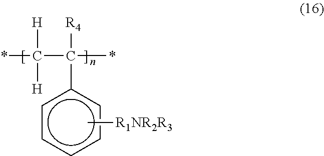

where n, R₁, R₂, R₃ and R₄ are defined above in the Formula (15).

A preferred form of the structure of the Formula (16) is shown below in the Formula (17):

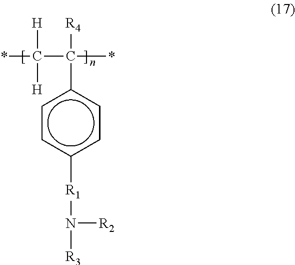

where the R₁NR₂R₃ group is located at the para-position, and where n, R₁, R₂, R₃ and R₄ are defined above in the Formula (15).

Another example of a hydrogen acceptor containing block that comprises a nitrogen containing group is shown below in the Formula (18)

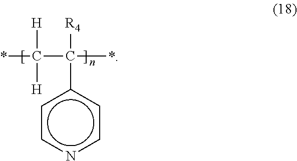

In the formula (4), n and R₄ are defined in Formula (15) and the nitrogen atom can be in the ortho, meta, para positions or any combination thereof (e.g., in both the ortho and para positions).

Yet another example of a hydrogen acceptor containing block that comprises a nitrogen containing group are shown below in the Formula (19)

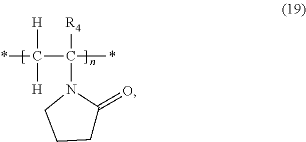

where n and R₄ are defined above in the Formula (15).

Yet another example of a hydrogen acceptor containing block that comprises a nitrogen containing group are poly(alkylene imines) shown below in the Formula (20)

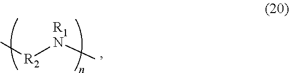

where R₁ is a 5 membered ring that is substituted with 1-4 nitrogen atoms, R₂ is a C₁ to C₁₅ alkylene and n represents the total number of repeat units. An example of the structure of Formula (20) is polyethyleneimine. Exemplary structures of the hydrogen acceptor of the Formula (20) are shown below.

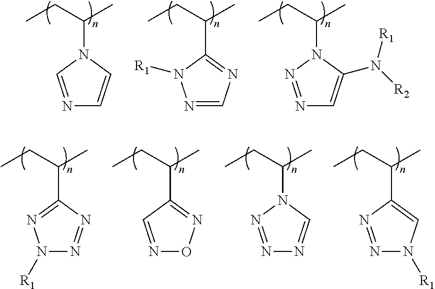

As noted above, the block comprising the hydrogen acceptor may be protected by a blocking group if desired. The hydrogen acceptor may be protected or blocked by an acid decomposable group, a thermally decomposable group or a group that can be decomposed by electromagnetic radiation. In an embodiment, the acid decomposable group can be thermally decomposed or decomposed as a result of exposure to electromagnetic radiation.

Examples of protected amine blocks (blocked or protected acceptors) are shown below in the formulas (18)-(21).

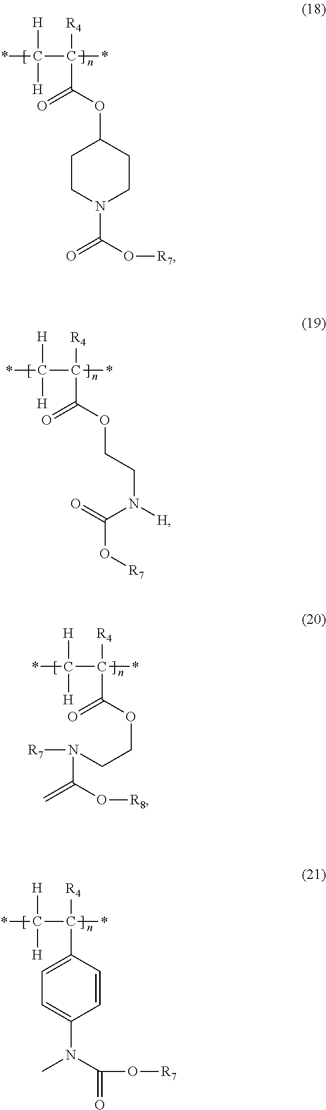

where in the applicable formulas (18) through (21), R₄ is a hydrogen or a C₁ to C₁₀ alkyl, R₇ and R₈ are the same or different and are independently a C₁ to C₃₀ alkyl group, and preferably a C₁ to C₁₀ group.

Exemplary hydrogen acceptor containing polymers used in the second polymeric layer 155B are poly(4-vinylpyrollidone), poly(2-vinylpyrollidone), copolymers of poly(4-vinylpyrollidone) and poly(2-vinylpyrollidone), and blends thereof.

The polymer used in the second polymeric layer 155B is a homopolymer having a weight average molecular weight average of 1,000 to 100,000 grams per mole, preferably 3,000 to 30,000 grams per mole.

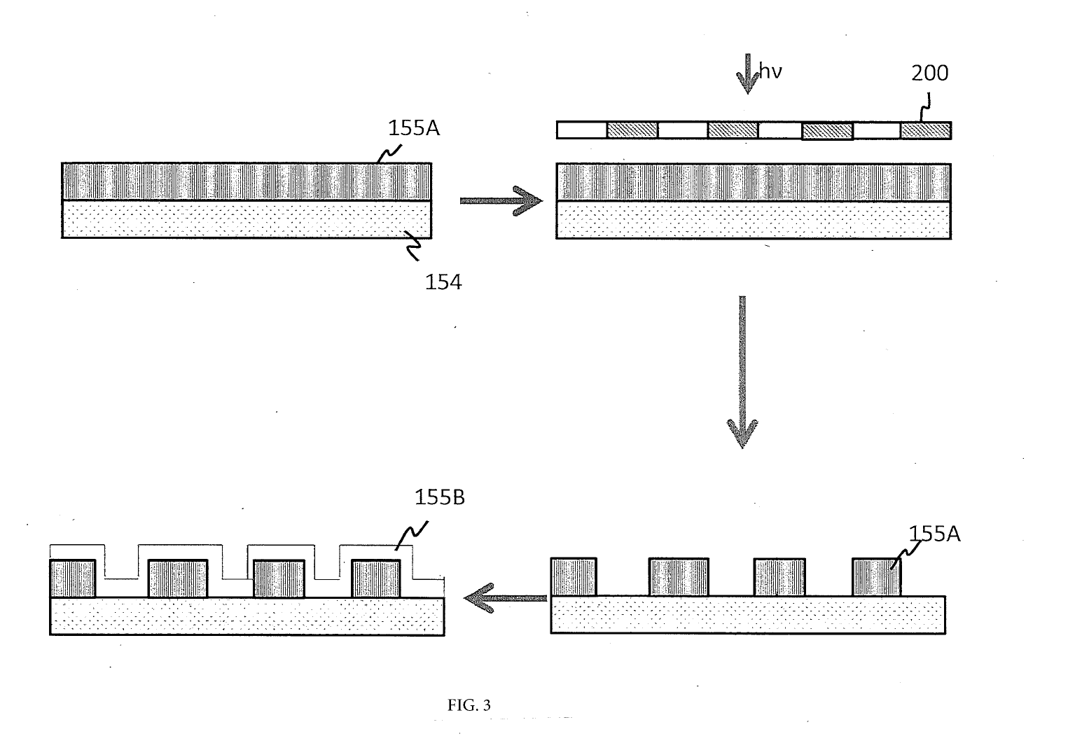

With reference now to the FIGS. 2(A), 2(B) and 3, in one manner of manufacturing the gas sensor, the first polymeric layer 155A is disposed on the substrate 154. The first polymeric layer 155A is part of a first composition that may contain in addition to the polymer, a solvent. The first composition is first disposed on the substrate 154. The first composition may then be dried by evaporating the solvent to form the first polymeric layer 155A having the first surface and the second surface. A photoresist may then be disposed on second surface of the first polymeric layer. Portions of the first polymeric layer 155A may then be etched to increase the surface area of the second surface. The second surface therefore has a textured surface that is at least twice the surface area of the first surface, preferably at least four times the surface of the first surface. This is depicted in the FIG. 3, where the first polymeric layer 155A is disposed on the surface of the substrate 154. The first polymeric layer may be disposed on the substrate using spin coating, spray painting, dip coating, doctor blading, or the like.

A photoresist 200 is then disposed on the second surface of the first polymeric layer 155A and portions of the first layer 155A are removed using radiation (hv), chemical etching, ion beam etching, or the like, to form a textured second surface. As seen in the FIG. 2(A), only a portion of the first polymeric layer 155A may be removed such that when the second polymeric layer 155B is disposed on the first polymeric layer 155A it contacts a surface of the first polymeric layer 155A along its entire area.

As seen in the FIG. 2(B) however, portions of the first polymeric layer 155A may be removed such that when the second polymeric layer 155B is disposed on the first polymeric layer 155A it contacts a surface of the first polymeric layer 155A along its entire area but in addition, contacts the substrate. In other words, portions of the first polymeric layer 155A can be etched away to expose a surface of the substrate 154.

After portions of the first polymeric layer 155A are removed, the protected hydrogen donor may be deprotected using electromagnetic radiation, thermal decomposition, a photoacid generator, an acid generator, or the like, or a combination thereof. The second polymeric layer 155B is then disposed on the second surface of the first polymeric layer 155A using spin coating, spray painting, dip coating, doctor blading, or the like.

The second polymeric layer 155B may be obtained by disposing on the first polymeric layer 155A a second composition that comprises a solvent as well as a polymer that contains a hydrogen acceptor. If the second polymeric layer 155B contains a protected hydrogen acceptor, it may be deprotected using electromagnetic radiation, thermal decomposition, a photoacid generator, an acid generator, or the like, or a combination thereof. As may be seen in the FIG. 3, the free surface of the second polymeric layer 155B has a higher surface area than the first surface (which is the surface that contacts the substrate) of the first polymeric layer 155A.

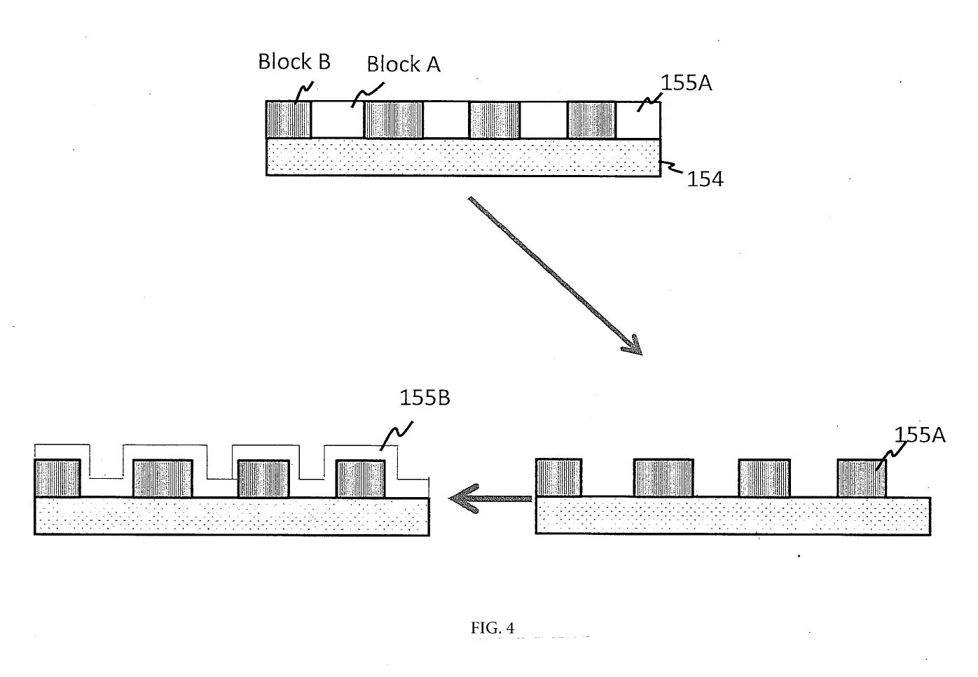

If the polymer used in the first polymeric layer 155A is a copolymer, the first polymer and the second polymer may phase separate (into phase A produced by Block A and phase B produced by Block B) upon being disposed on the substrate 154. This is shown in the FIG. 4. One of the phases of the block copolymer may be etched to form the textured second surface upon which the second polymeric layer 155B is disposed. The first and the second polymeric layers may be subjected to baking if desired. Suitable baking temperatures are from 75 to 200° C., preferably 100 to 150° C.

The total thickness of the first and the second polymeric layer is about 10 to 3000 nanometers, preferably 100 to 1500 nanometers. The thickness of the layers provides the ability to manufacture small and light weight gas sensors.

Suitable solvents that may be used in the respective first and second compositions include, for example: alkyl esters such as n-butyl acetate, n-butyl propionate, n-pentyl propionate, n-hexyl propionate and n-heptyl propionate, and alkyl butyrates such as n-butyl butyrate, isobutyl butyrate and isobutyl isobutyrate; ketones such as 2-heptanone, 2,6-dimethyl-4-heptanone and 2,5-dimethyl-4-hexanone; aliphatic hydrocarbons such as n-heptane, n-nonane, n-octane, n-decane, 2-methylheptane, 3-methylheptane, 3,3-dimethylhexane and 2,3,4-trimethylpentane, and fluorinated aliphatic hydrocarbons such as perfluoroheptane; and alcohols such as straight, branched or cyclic C₄-C₉ monohydric alcohol such as 1-butanol, 2-butanol, 3-methyl-1-butanol, isobutyl alcohol, tert-butyl alcohol, 1-pentanol, 2-pentanol, 1-hexanol, 1-heptanol, 1-octanol, 2-hexanol, 2-heptanol, 2-octanol, 3-hexanol, 3-heptanol, 3-octanol and 4-octanol; 2,2,3,3,4,4-hexafluoro-1-butanol, 2,2,3,3,4,4,5,5-octafluoro-1-pentanol and 2,2,3,3,4,4,5,5,6,6-decafluoro-1-hexanol, and C₅-C₉ fluorinated diols such as 2,2,3,3,4,4-hexafluoro-1,5-pentanediol, 2,2,3,3,4,4,5,5-octafluoro-1,6-hexanediol and 2,2,3,3,4,4,5,5,6,6,7,7-dodecafluoro-1,8-octanediol; toluene, anisole and mixtures containing one or more of these solvents. Of these organic solvents, alkyl propionates, alkyl butyrates and ketones, preferably branched ketones, are preferred and, more preferably, C₈-C₉ alkyl propionates, C₈-C₉ alkyl propionates, C₈-C₉ ketones, and mixtures containing one or more of these solvents. Suitable mixed solvents include, for example, mixtures of an alkyl ketone and an alkyl propionate such as the alkyl ketones and alkyl propionates described above. The solvent component of the first composition or the second composition is typically present in an amount of from 75 to 99 wt % based on the total weight of the first composition or the second composition.

When the sensing element is contacted by the fluid, certain gaseous molecules in the fluid contact the second polymeric layer and bond to it. The weight difference of the sensing element prior to and after the bonding of the gaseous molecules results in the generation of a proportional electrical current by the piezoelectric substrate. The electrical signal is calibrated to indicate to the user the molecule(s) that have interacted with the second polymeric layer 155B. The increased surface area of the free surface of the second polymeric layer 155B facilitates an increased collection of hazardous gas molecules on the surface of the sensing element, thus increasing the sensitivity of the gas sensor.

The gas sensor may thus be used to detect hazardous gases that are present in the environment in residential, commercial or industrial environments. In particular, the gas sensor is used in refrigerators, appliances and storage areas where food products and perishable items may be stored. The gas sensor may be used to detect hazardous, unwanted or disagreeable gases such as carbon monoxide, carbon dioxide, formaldehyde, hydrogen sulfide, amines, ozone, ammonia, benzene, and so on.

Another application for the gas sensor lies in an analysis of the breath or volatile gases emitted by biological processes, or for the diagnosis of diseases. For example, human breath contains a number of volatile organic compounds (VOCs). Accurate detection of VOCs in exhaled breath can provide essential information for the early diagnosis of diseases. For example, acetone, hydrogen sulfide, ammonia, mercaptans, nitrogen monoxide and toluene can be used to evaluate diabetes, halitosis, kidney malfunction, and lung cancer, respectively, where the diagnosis of these diseases can be achieved by analyzing the concentration of VOCs in exhaled breath, originating from the molecular exchange between lung tissue and blood. Variations in the concentration of the exhaled VOCs that may serve as biomarkers for specific diseases can distinguish healthy people from those who are sick.

Another application of gas sensor detection of gases may be for the monitoring the ripening of foodstuffs such as fruits, or the over ripening of fruits, or the aging or decay of foodstuffs such as fish and meat products. For example, ripening fruit generates ethylene gas. Accurate detection of ethylene gas or other volatile gases emitted by a fruit could monitor shelf life or peak ripeness. Aging or spoiling of fish products generates amines such as trimethylamine, hydrogen sulfide, sulfur dioxide, nitrogen oxides, and ammonia, and aging or spoiling of meats generates other volatile components such as ethyl acetate, methane, carbon dioxide and ammonia. Variations in the concentration of the emitted VOCs may be used to diagnose the usefulness, quality and safety of the products.

Another application of gas sensor detection of exhaled gases may be for the monitoring the blood alcohol level of a human being for the purposes of safe operation of equipment such as cars, trucks, boats and airplanes or other industrial equipment. In addition, such gas sensor detection of exhaled gases could also have forensic or law enforcement applications. For example, variations in the concentration of alcohol, ketones and aldehydes in the breath is closely correlated with the blood alcohol level.

The gas sensor may exemplified by the following non-limiting example.

### Example

This is a paper example to demonstrate the viability of manufacturing a gas sensor that may be used for the detection of gases. The first layer 155A was actually experimentally synthesized, while the sensing layer 155B and the gas detection portion of this example are conceptual ideas as of the filing of this application. The disposing of the first layer 155A on the piezoelectric substrate, the etching of the first layer 155A and the disposing of the second layer 155B on an etched surface of the first layer 155A are all concepts that demonstrate that it is feasible to manufacture the gas sensor.

This example demonstrates the manufacturing of a patterned first polymeric layer 155A that interacts with the second polymeric layer 155B by non-covalent interaction. The following monomers were employed in the syntheses of patterned first polymeric layer. They are—1-ethylcyclopentyl methacrylate (ECPMA), 1-methylcyclopentyl methacrylate (MCPMA), 2-propenoic acid, 2-methyl-, 2-[(hexahydro-2-oxo-3,5-methano-2H-cyclopenta[b]furan-6-yl) oxy]-2-oxoethyl ester (MNLMA) and 3-hydroxy-1-adamantyl acrylate (HADA).

Monomers of ECPMA (5.092 grams (g)), MCPMA (10.967 g), MNLMA (15.661 g) and HADA (8.280 g) were dissolved in 60 g of propylene glycol monomethyl ether acetate (PGMEA). The monomer solution was degassed by bubbling with nitrogen for 20 minutes. PGMEA (27.335 g) was charged into a 500 mL three-neck flask equipped with a condenser and a mechanical stirrer and was degassed by bubbling with nitrogen for 20 minutes. Subsequently, the solvent in the reaction flask was brought to a temperature of 80° C. V601 (dimethyl-2,2-azodiisobutyrate) (0.858 grams) was dissolved in 8 grams of PGMEA and the initiator solution was degassed by bubbling with nitrogen for 20 minutes. The initiator solution was added into the reaction flask and then monomer solution was fed into the reactor dropwise over a 3 hour period under rigorous stirring and nitrogen environment. After monomer feeding was complete, the polymerization mixture was left standing for one additional hour at 80° C. After a total of 4 hours of polymerization time (3 hours of feeding and 1 hour of post-feeding stirring), the polymerization mixture was allowed to cool down to room temperature. Precipitation was carried out in methyl tert-butyl ether (MTBE) (1634 g).

The powder precipitated was collected by filtration, air-dried overnight, re-dissolved in 120 g of THF, and re-precipitated into MTBE (1634 g). The final polymer was filtered, air-dried overnight and further dried under vacuum at 60° C. for 48 hours to give 31.0 grams of poly(ECPMA/MCPMA/MNLMA/HADA) (15/35/30/20) copolymer (MP-1) (Mw=20,120 grams per mole and Mw/Mn=1.59).

The final polymer poly(ECPMA/MCPMA/MNLMA/HADA) is then dissolved in a solvent and disposed on a piezoelectric substrate that comprises quartz to form the first polymeric layer 155A. A mask 200 (see FIG. 3) is disposed on the first layer 155A and portions of the first layer 155A that are not cured are removed by dissolution. The protected ester moieties from the first polymeric layer 155A are then deprotected by exposure to an acid, an acid generator or by exposure to an elevated temperature that promotes degradation of the ester moiety. The deprotection exposes the hydrogen donor and eventually facilitates an interaction with the second polymeric layer 155B. The dissolution of portions of the first polymeric layer 155A causes an increase in the surface area of the first layer 155A (the surface that does not contact the substrate).

A second polymeric layer 155B that comprises a poly(4-vinylpyrollidone) hydrogen acceptor is then cast onto the textured surface of the first polymeric layer 155A. After drying the second polymeric layer 155B, the gas sensor comprising the piezoelectric substrate 154, the first polymeric layer 155A with the textured surface contacting the second polymeric layer 155B is placed in contact with the appropriate electronics. The device is placed in a stream containing a trace of acetic acid. An increase in the weight of the sensor is detected by virtue of an electrical current generated by the piezoelectric substrate.

The sensor can therefore be used to detect acidic molecules that are present in the ambient atmosphere around the sensor. In one embodiment, the sensor can detect the present of undesirable or disagreeable molecules (in the atmosphere) by a weight difference of the sensor prior to and after being exposed to undesirable or disagreeable gases. In another embodiment, the sensor can detect the presence of undesirable or disagreeable molecules by means of a difference in electrical conductivity of the sensing layer 155B prior to and after being exposed to undesirable or disagreeable gases. In yet another embodiment, the sensor can detect the presence of undesirable or disagreeable molecules by virtue of a chemical analysis of molecules disposed on the sensing surface prior to and after being exposed to undesirable or disagreeable gases.

The sensor can also be provided with capabilities to replenish or refurbish the sensing surface after it has been expended in detecting various molecules of disagreeable or undesirable gases. In one embodiment, the sensor may be chemically treated to refurbish the contaminated sensor surface. In another embodiment, the sensor can be heated to a temperature effective to refurbish the contaminated surface by causing the detected gas molecules to debond from the surface. The heating to refurbish the surface can be conducted by conduction, radiation or convection.

What is claimed is:

1. A gas sensor comprising:
a substrate;
a first polymeric layer having a first surface and a second surface disposed on the substrate; where the first surface contacts the substrate and where the second surface is opposed to the first surface and has a higher surface area than the first surface; where the first polymeric layer comprises repeat units that have a deprotected hydrogen donor; and
a second polymeric layer disposed on the first polymeric layer; where the second polymeric layer is derived from a repeat unit that comprises a hydrogen acceptor.

2. The gas sensor of claim 1, wherein the repeat unit that comprises the hydrogen acceptor comprises a nitrogen-containing group and where the hydrogen acceptor is operative to undergo hydrogen bonding, van der Waals interactions, π-π interactions, electrostatic interactions, or a combination thereof, with a hydrogen donor of first polymer.

3. The gas sensor of claim 1, wherein the repeat unit that comprises the hydrogen acceptor further comprises a sensing acceptor; where the sensing acceptor is operative to undergo hydrogen bonding, van der Waals interactions, π-π interactions, electrostatic interactions, or a combination thereof, with a gas.

4. The gas sensor of claim 2, wherein the nitrogen-containing group is chosen from amine, amide and pyridine groups.

5. The gas sensor of claim 1, where the deprotected hydrogen donor is obtained by deprotecting repeat units that have a protected acid group and/or a protected alcohol group.

6. The gas sensor of claim 1, where the second surface is textured.

7. The gas sensor of claim 1, where the second surface that is textured has a surface area that is at least two times greater than a surface area of the first surface.

8. The gas sensor of claim 1, where the second polymeric layer has a free surface that is textured.

9. A method of manufacturing a gas sensor comprising:
disposing upon a substrate a first polymeric layer having a first surface and a second surface; where the first surface contacts the substrate and where the second surface is opposed to the first surface and has a higher surface area than the first surface; where the first polymeric layer comprises repeat units that have a deprotected hydrogen donor; and
disposing upon the first polymeric layer a second polymeric layer; where the second polymeric layer is derived from a repeat unit that comprises a hydrogen acceptor.

10. A method of detecting a gas comprising:
contacting a gas sensor with a gaseous molecule; where the gas sensor comprises:
a substrate;
a first polymeric layer having a first surface and a second surface disposed on the substrate; where the first surface contacts the substrate and where the second surface is opposed to the first surface and has a higher surface area than the first surface; where the first polymeric layer comprises repeat units that have a deprotected hydrogen donor; and
a second polymeric layer disposed on the first polymeric layer; where the second polymeric layer is derived from a repeat unit that comprises a hydrogen acceptor; and
forming at least one of a hydrogen bond, a van der Waals interaction, a π-π interaction or an electrostatic interaction between the gas molecule and the second polymeric layer; and
determining an identity of the gas molecule based on a difference of the sensor prior to and after the forming of the hydrogen bond, the van der Waals interaction, the π-π interaction and the electrostatic interaction.

11. The method of claim 10, where the difference is a weight difference or a difference in electrical conductivity.
